(19) United States
(12) Patent Application Publication
(10) Pub. No.: US 2006/0199847 A1
(43) Pub. Date: Sep. 7, 2006

(54) Stable ammonium salts of alpha-liponic acid, the production thereof and the use of the same

(76) Inventors: Martin Jochen Klatt, Bad Durkheim (DE); Frank Haunert, Mannheim (DE); Klaus Kramer, Landau (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/552,964

(22) PCT Filed: Apr. 14, 2004
(86) PCT No.: PCT/EP04/03958

§ 371 Date: Oct. 14, 2005

(30) Foreign Application Priority Data
Apr. 17, 2003 (DE) 10318045.1

(51) Int. Cl. A61K 31/4436; C07D 409/02; A61K 31/385; C07D 339/02

(52) U.S. Cl. 514/342; 546/280.7; 514/440; 549/35

(57) ABSTRACT

The invention relates to ammonium salts of α-liponic acid of general formula (I): (Lp) (A), formed from α-liponic acid (Lp) and amino compounds (A)=of general formula (II) or general formula (III). The invention also relates to a method for producing said salts, and to the use of said salts as constituents in pharmaceutical, dermatological and cosmetic agents, foodstuffs, animal feed supplements or food supplements. The invention also relates to pharmaceutical, dermatological and cosmetic agents, foodstuffs, animal feed supplements or food supplements in which the inventive salts are used.

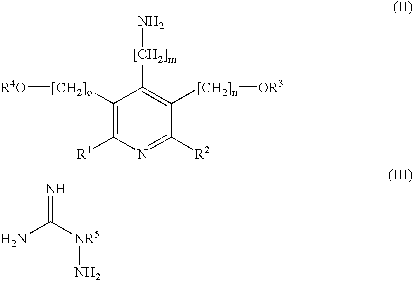

The term α-lipoic acid means hereinafter racemic α-lipoic acid or racemic dihydro-α-lipoic acid, the enantiomers (R)- or (S)-α-lipoic acid, (R)- or (S)-dihydro-α-lipoic acid, and all mixtures of the respective enantiomeric forms (R) and (S).

The present invention relates to ammonium salts of α-lipoic acid of the general formula I
(Lp)(A) I
where
Lp is α-lipoic acid and
A is an amine of the general formula II 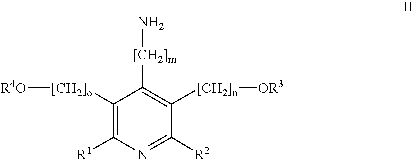
in which
R¹, R² are hydrogen, C₁- to C₆-alkyl,
R³, R⁴ are hydrogen, C₁- to C₈-alkyl, C₁- to C₈-acyl, phosphate, diphosphate, triphosphate
m, n, o are 0, 1, 2, 3,
   or
   A is an amine of the general formula III 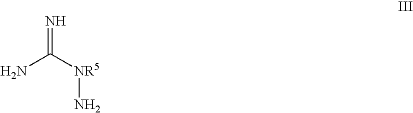
in which
R⁵ is hydrogen, C₁- to C₈-alkyl, phenyl, benzyl,
   and to processes for preparing (Lp)(A), to the use of (Lp)(A) as components of human foods, animal or human dietary supplements, in pharmaceutical and dermatological compositions and cosmetic formulations, and to these human foods, animal or human dietary supplements, pharmaceutical and dermatological compositions and cosmetic formulations themselves.

α-Lipoic acid acts as a coenzyme in the oxidative decarboxylation of pyruvate and other α-keto acids and is present in the form of its (R) enantiomer in virtually every cell of plant and animal organisms.

α-Lipoic acid is employed therapeutically for the treatment of liver disorders and for diabetic and alcoholic polyneuropathy, a change in peripheral nerves which is associated with metabolic disorders. Anti-inflammatory, analgesic and cytoprotective properties, as well as the antioxidant effect, make lipoic acid an interesting active ingredient for pharmacy, cosmetics, nutritional science and adjacent areas. Thus, Stoll et al. reported in Pharmacology Biochemistry and Behavior, Vol. 46, pp. 799-805 (1993) and in Ann. NY Acad. Sci., Vol. 717, pp. 122-128 (1994) that lipoic acid is able to improve the long-term memory of old mice and cognitive abilities of rodents.

T. M. Hagen et al. describe in FASEB-Journal, Vol. 13, pp. 411-418 (1999) a revitalizing effect of lipoic acid administered orally to old rats.

According to EP-A 0 947 194, the R enantiomer has mainly anti-inflammatory activity, while the S enantiomer has mainly antinociceptive activity. Overall, the optical isomers of α-lipoic acid are more active than the racemate.

The cyclic disulfide of α-lipoic acid can be converted in redox reactions into dihydrolipoic acid, the open-chain, reduced form. It acts as acyl donor in the pyruvate dehydrogenase complex of the mitochondrial membrane. It acts as antioxidant and is hydrogen donor in the reduction of α-keto acids. In the enzyme association, it is bound as amide to the F-amino group of a lysine residue.

In addition, α-lipoic acid or α-dihydrolipoic acid are able to increase the bioavailability of mineral salts (EP-A 1 172 110).

Pyridoxamine (4-aminomethyl-5-hydroxymethyl-2-methylpyridin-3-ol) forms together with pyridoxole, pyridoxal, pyridoxal phosphate and pyridoxamine phosphate the group of naturally occurring forms of vitamin B6.

Vitamin B6 is the most important coenzyme of amino acid metabolism.

Proteins with a long turnover time are exposed to chemical damage (aging) which is detectable in the form of so-called AGEs (advanced glycation end products) and ALEs (advanced lipoxidation end products).

AGEs are thought to be associated with many age-related disorders, including pathophysiological changes of retinal function (Hammes et al., Diabetologia vol. 42, pp. 728-736 (1999)) and dementias such as Alzheimer's disease.

Nucleophilic AGE inhibitors such as pyridoxamine and the guanidine derivative aminoguanidine, which trap reactive carbonyls and therefore inhibit the AGE formation associated with diabetes, also trap bioactive lipids and ALE precursors associated with arteriosclerosis.

Stitt et al. (Diabetes, vol. 51, pp. 2826-2831 (2002)) were able to show that pyridoxamine protects against a whole series of pathological changes of the retina associated with diabetes and can therefore be employed for the treatment of diabetic retinopathy.

It has additionally been possible to show that pyridoxamine inhibits the development of renal disorders (albuminuria, creatinemia) in diabetic rats (Degenhardt et al., Kidney Int, vol. 61(3), pp. 939-950 (2002)).

In contrast to aminoguanidine, although pyridoxamine does not act as antioxidant, because it does not prevent the peroxidation of lipids, it does inhibit the formation of malonaldehyde and 4-hydroxynonenal adducts and thus the chemical alteration of proteins.

Aminoguanidine reacts with dicarbonyl compounds such as methylglyoxal and 3-deoxyglucosone, which are known to be neurotoxic substances, and thus prevents the apoptosis of nerve cells.

Lipid oxidation leads to the formation of reactive α-unsaturated aldehydes such as 4-hydroxynonenal, acrolein and malonaldehyde, which lead to the ALEs after reaction with proteins.

Aminoguanidine is able to trap such compounds (Dukic-Stefanovic et al., Biogerontology vol. 2, pp. 19-34 (2001)).

EP-B 702 953 and EP-A 947 194 describe dosage forms of solid salts of α-lipoic acid, which are used as pharmaceutical or food additive.

According to EP-B 702 953, dosage forms of solid salts show increased bioavailability and easier producibility than dosage forms of the free acid.

The easier producibility is based on the fact that some salts tolerate, in contrast to the free acid, increases in temperature occurring locally for example during tableting. Since such temperature effects cannot be precluded in the production of many dosage forms, a thermally stable salt form has considerable advantages.

However, most α-lipoic acid salts show extremely low thermal stability.

Explicitly, trometamol (EP-A 947 194), sodium hydroxide (EP-A 947 194) and zinc nitrate (EP-A 1172110) are cited as salt formers of stable salts.

The invention was based on the object of preparing a further, easily obtainable, thermally stable salt of α-lipoic acid.

EP-A 0 572 922 discloses that combinations of the R enantiomer of α-lipoic acid and vitamins show increased activity compared with the effect of the racemic form of α-lipoic acid alone and the effect of the vitamins alone, i.e. have synergistic effects.

EP-A-0 572 922 describes the use of α-lipoic acid and derivatives thereof in combination with a vitamin for producing medicaments having analgesic, anti-inflammatory, antidiabetic, cytoprotective, anti-ulcerative, antinecrotic, neuroprotective, detoxifying, anti-ischemic, liver function-regulating, anti-allergic, immunostimulating and anti-oncogenic effects.

In this case, α-lipoic acid is combined with vitamins by preparing mixtures of the individual components.

The α-lipoic acid is in this case employed in the form of the free acid or in the form of its salts.

In the case of combinations of vitamins and α-lipoic acid it is necessary first to mix the suitable form of α-lipoic acid and the vitamin before a dosage form can be produced.

An operational step of preparing the mixture is therefore initially necessary for producing a dosage form intended to comprise a combination of α-lipoic acid and vitamin.

It is therefore preferred according to the invention to provide a stable α-lipoic acid salt of a second component which has therapeutic or cosmetic activity or is suitable as addition to human foods or human or animal dietary supplements with the intention of dispensing with the operational step of preparing a mixture of the components.

This object has been achieved by the provision of the compounds I mentioned at the outset.

The compounds I of the invention permit simultaneous administration of α-lipoic acid and of a second component which has therapeutic or cosmetic activity or can be used as addition to human foods or human or animal dietary supplements.

Preferred as second component are compounds of the general formula II in which R¹ and R² are independently of one another hydrogen or C₁- to C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 1,1,2-trimethylpropyl, 1,2,2-trimethylpropyl, 1-ethyl-1-methylpropyl, 1-ethyl-2-methylpropyl, in particular C₁- to C₄-alkyl, preferably hydrogen or methyl.

R³ and R⁴ are independently of one another hydrogen, C₁- to C₆-alkyl as mentioned in detail above, heptyl, octyl, the corresponding acyl radicals and mono-, di-, triphosphate.

R³ is preferably hydrogen or methyl, particularly preferably hydrogen. R⁴ is preferably hydrogen or methyl, particularly preferably hydrogen.

The indices m, n or o in formula II are integers from 0 to 3, and one of the indices n or o is preferably not zero. It is particularly preferred for n to be one and o to be zero. m is preferably one.

Amines of the formula II which may be mentioned by way of example are listed in

TABLE 1

| R¹ | R² | R³ | R⁴ | m | n | o |
|---|---|---|---|---|---|---|
| Methyl | H | H | H | 1 | 1 | 0 |
| Ethyl | H | H | H | 1 | 1 | 0 |
| Propyl | H | H | H | 1 | 1 | 0 |
| 1-Methylethyl | H | H | H | 1 | 1 | 0 |
| Butyl | H | H | H | 1 | 1 | 0 |
| 1-Methylpropyl | H | H | H | 1 | 1 | 0 |
| 2-Methylpropyl | H | H | H | 1 | 1 | 0 |
| 1,1-Dimethylethyl | H | H | H | 1 | 1 | 0 |
| Methyl | Methyl | H | H | 1 | 1 | 0 |
| Methyl | Ethyl | H | H | 1 | 1 | 0 |
| Methyl | Propyl | H | H | 1 | 1 | 0 |
| Methyl | 1-Methylethyl | H | H | 1 | 1 | 0 |
| Methyl | Butyl | H | H | 1 | 1 | 0 |
| Methyl | 1-Methylpropyl | H | H | 1 | 1 | 0 |
| Methyl | 2-Methylpropyl | H | H | 1 | 1 | 0 |
| Methyl | 1,1-Dimethylethyl | H | H | 1 | 1 | 0 |

Pyridoxamine is particularly preferred according to formula II.

Also suitable according to the invention as salt former is an optionally substituted aminoguanidine of the formula III.

R⁵ in formula III is hydrogen, C₁- to C₆-alkyl as mentioned in detail above, phenyl or benzyl. R₅ is preferably hydrogen, and aminoguanidine is particularly preferred according to formula II.

It has surprisingly been found that the salts of the invention have sufficient stability and can be prepared by a cost-effective process.

The preferred form of α-lipoic acid is R-α-lipoic acid and mixtures of R- and S-α-lipoic acid, where the ratio of the amounts of R form and S form is greater than 1, e.g. R/S is 70/30.

The invention further relates to processes for preparing the salts of the general formula I from α-lipoic acid and amines of the general formula II or III in a solvent at a temperature of from 40 to 80° C. and isolating the solid in a manner known per se. The required product is expediently isolated by cooling the reaction mixture until crystallization starts, and then filtering off the salt.

Preferred solvents are protic solvents, in particular alcohols, particularly preferably methanol, ethanol, propanol, isopropanol, very particularly preferably ethanol.

A filtration aid can be employed to improve separation, such as, for example, silica gel.

The salt is normally dried after the isolation.

The invention further relates to the use of the salts of the formula I as component in human foods, animal or human dietary supplements, for producing dermatological compositions, in cosmetic formulations and pharmaceuticals.

The cosmetic and dermatological compositions are intended preferably to prevent damage to the skin and hair and/or unwanted changes in the appearance of the skin.

They are intended in particular to be suitable for the treatment of damage to the skin and hair and unwanted changes in the appearance of the skin which have already occurred.

In this connection, the use can take place both in cosmetic compositions such as bodycare compositions, decorative cosmetics etc., which are usually available without prescription, and in dermatological compositions, meaning medicaments for the therapy of disorders of the skin (dermatoses). Dermatological compositions may additionally comprise at least one further active ingredient which is preferably selected from antimycotics, antiseptics, antibiotics, sulfonamides, disinfectants, corticoids, shale oil sulfonates and tar sulfonates, astringents, antihydrotics, remedies for acne, psoriasis, seborrhea and pruritus, keratolytics etc.

The preparations may comprise cosmetic excipients normally used in such preparations, e.g. preservatives, bactericides, perfumes, substances for preventing foaming, colorants, pigments, thickeners, surface-active substances, emulsifiers, emollient substances, hardening agents, fats, oils, waxes or other usual ingredients of a cosmetic or dermatological formulation such as alcohols, polyols, polymers, foam stabilizers, solubilizers, electrolytes, organic acids, organic solvents or silicone derivatives.

The preparations may comprise, in addition to the active ingredients mentioned, further compounds which act as antioxidants, as radical scavengers, moisturizers or moisture retainers, or have anti-erythematous, anti-inflammatory or anti-allergic effects, in order to supplement or enhance the effect thereof. These compounds can be selected in particular from the group of vitamins, plant extracts, α- and β-hydroxy acids, ceramides, anti-inflammatory, antimicrobial or UV-filtering substances, and their derivatives and mixtures thereof. The antioxidants are advantageously selected from amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotenoids, carotenes (e.g. α-carotene, β-carotene, lycopene) and their derivatives, chlorogenic acid and its derivatives, aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated dosages (e.g pmol to pmol/kg), also (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. y-linolenic acid, linoleic acid, oleic acid), folic acid and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and its derivatives, butylhydroxytoluene, butylhydroxyanisole, norihydroguajak resin acid, nordihydroguajaretic acid, trihydroxybutyrophenone, uric acid and its derivatives, mannose and its derivatives, sesamol, sesamolin, zinc and its derivatives (e.g. ZnO, ZnSO4), selenium and its derivatives (e.g. selenomethionine), stilbene and its derivatives (e.g. stilbene oxide, trans-stilbene oxide) and the derivatives suitable according to the invention (salts, esters, ethers, sugars, nucleotides, nucleosides, peptides and lipids) of these active ingredients mentioned.

The cosmetic and dermatological preparations preferably additionally comprise substances which absorb UV radiation in the UV-B and/or UV-A range. Examples of suitable UV filters are 2,4,6-triaryl-1,3,5-triazines in which the aryl groups may each have at least one substituent which is preferably selected from hydroxy, alkoxy, specifically methoxy, alkoxycarbonyl, specifically methoxycarbonyl and ethoxycarbonyl, and mixtures thereof. Also suitable are 4-aminobenzoic esters where the amino group may be alkylated or alkoxylated, if appropriate. These include for example isooctyl N,N-dimethyl-4-aminobenzoate. Also suitable are 2-hydroxybenzoic esters such as, for example, the isooctyl ester. Further suitable UV filters are 2,4,6-trianiline(o-carbo-2′-ethylhexyl-1′-oxy)-1,3,5-triazine, 3-imidazol-4-ylacrylic acid and its ethyl ester, menthyl o-aminobenzoate, glyceryl p-aminobenzoate, 2,2′-dihydroxy-4-methoxybenzophenone (dioxybenzone), 2-hydroxy-4-methoxy-4-methylbenzophenone-triethanolamine salicylate, dimethoxyphenylglyoxalic acid, 3-(4′sulfo)benzylidenebornan-2-one and its salts, 2,2′,4,4′-tetrahydroxybenzophenone, 2,2′-methylenebis-[6(2H-benzotriazol-2-yl-4-(1,1,3,3,-tetramethylbutyl)phenol], 2,2′-(1,4-phenylene)bis-1H-benzimidazole-4,6-disulfonic acid and its sodium salt, 2,4-bis[4-(2-ethylhexyloxy)-2-hydroxy]phenyl-6-(4-methoxyphenyl)-(1,3,5)-triazine, 3-(4-methylbenzylidene)camphor, 4-bis(polyethoxy)para-aminobenzoic acid polyethoxyethyl ester, 2,4-dihydroxybenzophenone and/or 2,2′-dihydroxy-4,4′-dimethoxybenzophenone-5,5′-disodium sulfonate.

The invention also relates to the production of compositions for the treatment of an individual, preferably of a mammal, in particular of a human, agricultural or domestic animal.

The present invention therefore also relates to compositions comprising the compounds of the invention of the general formula 1, if appropriate at least one further active ingredient and a formulation base.

The compositions include cosmetics, dermatological compositions, medicaments, human foods, animal or human dietary supplements comprising salts of the formula 1.

The human foods and dietary supplements of the invention have, besides the nutrition-related function, additionally an active ingredient-related function. They are therefore referred to as functional foods and dietary supplements.

The formulation base of formulations of the invention comprises physiologically acceptable excipients. Physiologically acceptable excipients are those known to be usable in the area of pharmacy, of food technology and adjacent areas, in particular those listed in relevant pharmacopoeias (e.g. DAB, Pi. Euer., BP, NF) and also other excipients whose properties do not stand in the way of physiological use. Excipients for the purposes of the invention may also have a nutritional value and therefore be used generally as food component. Essential nutrients may also be included.

Suitable excipients may be: lubricants, wetting agents, emulsifying and suspending agents, preserving agents, antioxidants, anti-irritants, chelating agents, tablet-coating aids, emulsion stabilizers, film formers, gel formers, masking odors, taste-masking agents, resins, hydrocolloids, solvents, solubilizers, neutralizers, permeation promoters, pigments, quaternary ammonium compounds, refatting and superfatting agents, ointment, cream or oil bases, silicone derivatives, spreading aids, stabilizers, sterilizers, suppository bases, tablet excipients such as binders, fillers, lubricants, disintegrants or coatings, propellants, desiccants, opacifiers, thickeners, waxes, plasticizers, white oils. An arrangement concerning this is based on expert knowledge as described for example in Fiedler, H. P., Lexikon der Hilfsstoffe für Pharmazie, Kosmetik und angrenzende Gebiete, 4th edition, Aulendorf: ECV-Editio-Kantor-Verlag, 1996.

Food components generally comprise one or more amino acids, carbohydrates or fats and are suitable for human and/or animal nutrition. They comprise individual components, frequently vegetable but also animal products, especially sugars, if appropriate in the form of syrups, fruit preparations such as fruit juices, nectar, fruit pulps, purees or dried fruits, for example apple juice, grapefruit juice, orange juice, apple puree, tomato sauce, tomato juice, tomato puree, cereals products such as wheat flour, rye flour, oat flour, corn flour, barley flour, spelt flour, corn syrup, and starches from said cereals; dairy products such as milk protein, whey, yoghurt, lecithin and lactose. Typical examples of food components are infant food, breakfast preparations, especially in the form of mueslis or bars, sports drinks, complete meals, especially forming part of completely balanced diets which can be administered orally or enterally, dietary products such as diet drinks, diet meals and diet bars.

The essential nutrients include in particular vitamins, provitamins, minerals, trace elements, amino acids and fatty acids. Essential amino acids which may be mentioned are isoleucine, leucine, lysine, methionine, phenylalanine, threonine, tryptophan and valine. Also included are semi-essential amino acids which must be given, for example, in periods of growth or deficiency states, such as glutamine, arginine, histidine, cysteine and tyrosine. Trace elements which may be mentioned are: essential trace elements and minerals which have proved to be necessary for humans, and deficiency of which leads to manifestation of clinical symptoms: iron, copper, zinc, chromium, selenium, calcium, magnesium, potassium, manganese, cobalt, molybdenum, iodine, silicon, fluorine. Likewise elements whose function in humans is as yet inadequately verified: tin, nickel, vanadium, arsenic, lithium, lead, boron. Fatty acids essential for humans which may be mentioned are: linoleic acid and linolenic acid, ARA (arachidonic acid) and DHA (docosahexaenoic acid) for babies and possibly EPA (eicosapentaenoic acid) and DHA also for adults. A comprehensive list of vitamins is to be found in “Referenzwerte für die Nährstoffzufuhr”, 1st edition, Umschau Braus Verlag, Frankfurt am Main, 2000, edited by the Deutschen Gesellschaft für Ernährung.

Examples of suitable pharmaceutical formulations are solid drug forms such as oral powders, dusting powders, granules, tablets, especially film-coated tablets, pastilles, sachets, cachets, sugar-coated tablets, capsules such as hard and soft gelatin capsules, suppositories or vaginal drug forms, semisolid drug forms such as ointments, creams, hydrogels, pastes or patches, and liquid drug forms such as solutions, emulsions, especially oil-in-water emulsions, suspensions, for example lotions, preparations for injection and infusion, eye drops and ear drops. It is also possible to use implantable delivery devices for administering active ingredients of the invention. It is moreover possible to use liposomes or microspheres. In each case, the active ingredients may each be combined if appropriate with appropriate excipients and carriers.

Examples of suitable excipients and carriers are substances such as fillers, preservatives, tablet disintegrants, flow regulators, plasticizers, wetting agents, dispersants, emulsifiers, solvents, release-slowing agents or antioxidants. Examples of carriers and excipients are gelatin, natural sugars such as sucrose or lactose, lecithin, pectin, starch (for example corn starch or amylose), cyclodextrins and cyclodextrin derivatives, dextran, polyvinylpyrrolidone, polyvinyl acetate, gum arabic, alginic acid, Tylose, talc, lycopodium, silica, cellulose, cellulose derivatives (for example cellulose ethers in which the cellulose hydroxy groups are partly etherified with lower saturated aliphatic alcohols and/or lower saturated aliphatic oxy alcohols, for example methyloxypropylcellulose, methylcellulose, hydroxypropylmethylcellulose, hydroxypropylmethylcellulose phthalate); fatty acids, and magnesium, calcium or aluminum salts of fatty acids having 12 to 22 C atoms, especially saturated (for example stearates), emulsifiers, oils and fats, especially vegetable (for example peanut oil, castor oil, olive oil, sesame oil, cottonseed oil, corn oil, wheat germ oil, sunflower seed oil, cod liver oil, in each case also hydrogenated); glycerol esters and polyglycerol esters of saturated fatty acids C₁₂H₂₄0₂ to C₁₈H₃₆0₂ and mixtures thereof, where the glycerol hydroxyl groups are completely or else only partly esterified (for example mono-, di- and triglycerides); pharmaceutically suitable monohydric or polyhydric alcohols and polyglycols such as polyethylene glycols (molecular weights for example between 300 and 1500) and derivatives thereof, polyethylene oxide, esters of aliphatic saturated or unsaturated fatty acids (2 to 22 C atoms, especially 10 to 18 C atoms) with monohydric aliphatic alcohols (1 to 20 C atoms) or polyhydric alcohols such as glycols, glycerol, diethylene glycol, pentaerythritol, sorbitol, mannitol etc., which may also be etherified if appropriate, esters of citric acid with primary alcohols, acetic acid, urea, benzyl benzoate, dioxolanes, glycerol formals, tetrahydrofurfuryl alcohol, polyglycol ethers with C₁-C₁₂ alcohols, dimethylacetamide, lactamides, lactates, ethyl carbonates, silicones (especially medium-viscosity polydimethylsiloxanes), calcium carbonat, sodium carbonate, calcium phosphate, sodium phosphate, magnesium carbonate and the like.

Further suitable excipients are also so-called disintegrants (substances which bring about disintegration of the tablet), such as crosslinked polyvinylpyrrolidone (Kollidon® CL), sodium carboxymethylstarch, sodium carboxymethylcellulose or microcrystalline cellulose. It is likewise possible to use known covering materials such as polymers and copolymers of (meth)acrylic acid and/or esters thereof, copolymers of acrylic and methacrylic esters with a low content of ammonium groups (for example Eudragit0 RS), copolymers of acrylic and methacrylic esters and trimethylammonium methacrylate (for example Eudragito RL), polyvinyl acetate; fats, oils, waxes, fatty alcohols, hydroxypropylmethylcellulose phthalate or acetate succinate; cellulose acetate phthalate, starch acetate phthalate, and polyvinyl acetate phthalate, carboxymethylcellulose, methylcellulose phthalate, methylcellulose succinate, phthalate succinate and methylcellulose hemiester of phthalic acid, zein, ethylcellulose and ethylcellulose succinate, shellac, gluten, ethylcarboxyethylcellulose, ethacrylate-maleic anhydride copolymer, maleic anhydride-vinyl methyl ether copolymer, styrene-maleic acid copolymers, 2-ethylhexyl acrylate-maleic anhydride, crotonic acid-vinyl acetate copolymer, glutamic acid/glutamic ester copolymer, carboxymethylethylcellulose glycerol monocianoate, cellulose acetate succinate, polyarginine.

Further possible ingredients are plasticizers for covering materials such as citric and tartaric esters (acetyl triethyl citrate, acetyl tributyl, tributyl, triethyl citrates), glycerol and glycerol esters (glycerol diacetate, triacetate, acetylated monoglycerides, castor oil), phthalic esters (dibutyl, diamyl, diethyl, dimethyl, dipropyl phthalates), di-(2methoxy- or 2-ethoxyethyl)phthalate, ethyl phthalyl glycolate, butyl phthalyl ethyl glycolate and butyl glycolate, alcohols (propylene glycol, polyethylene glycol of various chain lengths), adipates (diethyl adipate, di(2-methoxy- or 2-ethoxyethyl)adipate), benzophenone, diethyl and dibutyl sebacates, dibutyl succinate, dibutyl tartrate, diethylene glycol dipropionate, ethylene glycol diacetate, dibutyrate, dipropionate, tributyl phosphate, tributyrin, polyethylene glycol sorbitan monooleate (polysorbates such as Polysorbat 80), sorbitan monooleate.

Suitable for preparing solutions or suspensions are, for example, water or physiologically tolerated organic solvents such as, for example, alcohols (ethanol, propanol, isopropanol, 1,2-propylene glycol, polyglycols and their derivatives, fatty alcohols, partial esters of glycerol) and oils (for example peanut oil, olive oil).

The pharmaceutical compositions comprising the compounds of the invention can be administered by oral, enteral, pulmonary, nasal, lingual, intravenous, intraarterial, intracardiac, intramuscular, intraperitoneal, intracutaneous or subcutaneous route or by inhalation.

The compounds of the invention have the advantage that α-lipoic acid and the second component which has pharmaceutical, dermatological or cosmetic activity or can be used in human foods or human and animal dietary supplements are present together in a stable formulation.

The inventive are therefore preferably suitable as space-saving ingredients in pharmaceutical, dermatological or cosmetic dosages and in human foods or animal and human dietary supplements, especially in solid dosage forms.

The inventive stable salts of lipoic acid allow α-lipoic acid and a second active component to be administered simultaneously.

### EXAMPLE

### Reaction of R-α-lipoic acid with pyridoxamine

1 mol of R-α-lipoic acid is dissolved in portions in ethanol at room temperature. 1 mol of pyridoxamine (dissolved in ethanol) is added while stirring. The mixture is heated to 50° C. and stirred at this temperature for 30 minutes. The solid is then filtered off under water pump vacuum, and the filter cake is washed with ethanol.

The clear filtrate is cooled under a nitrogen atmosphere until crystallization starts. The crystals are filtered off with suction under water pump vacuum and washed with ethanol. The crystalline solid is dried in a stream of nitrogen with exclusion of light.

Yield: 68% of theory

Melting point: 121-122° C.

1-15. (canceled)

16. A salt of α-lipoic acid of the general formula I
(Lp)(A) I
wherein
Lp is racemic α-lipoic acid, a racemic dihydro-α-lipoic acid, an (R)- or (S)-α-lipoic acid, an (R)- or (S)-dihydro-α-lipoic acid, or a mixture thereof; and
A is at least one amine of the general formula II 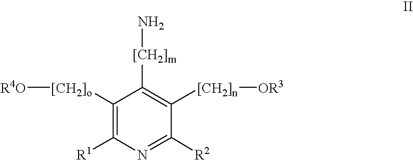
wherein
R¹ and R² are, independently, hydrogen or a C₁- to C₆-alkyl group;
R³ and R⁴ are, independently, hydrogen, a C₁- to C₈-alkyl group, a Cα- to C₈-acyl group, a phosphate group, a diphosphate group, or a triphosphate group; and
m and n and o independently are 0, 1, 2, or 3.

17. A salt of α-lipoic acid of the general formula I
(Lp)(A) I
wherein
Lp is racemic α-lipoic acid, a racemic dihydro-α-lipoic acid, an (R)- or (S)-α-lipoic acid, an (R)- or (S)-dihydro-α-lipoic acid, or a mixture thereof; and
A is at least one amine of the general formula III 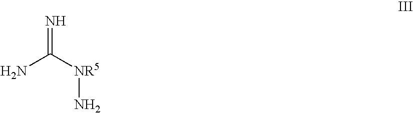
wherein
R⁵ is selected from the group consisting of hydrogen, a C₁- to C₆-alkyl group, a phenyl group, and a benzyl group.

18. The salt according to claim 16, wherein R¹ and R² independently are hydrogen or methyl.

19. The salt according to claim 16, where n is 1.

20. The salt according to claim 16, wherein either n or o is greater than zero.

21. The salt according to claim 16, wherein R³ is hydrogen.

22. The salt according to claim 16, wherein A is pyridoxamine.

23. The salt of the formula I according to claim 17, where A is aminoguanidine.

24. A process for preparing salts of o-lipoic acid of the general formula I according to claim 16, comprising
reacting racemic α-lipoic acid, racemic dihydro-α-lipoic acid, (R)- or (S)-α-lipoic acid, (R)- or (S)-dihydro-α-lipoic acid, or mixtures thereof with at least one amine of general formula II in solution at a temperature from 40 to 80° C.; and
isolating the thus prepared salts of α-lipoic acid.

25. A process for preparing salts of α-lipoic acid of the general formula I according to claim 17, comprising
reacting racemic o-lipoic acid, racemic dihydro-α-lipoic acid, (R)- or (S)-α-lipoic acid, (R)- or (S)-dihydro-α-lipoic acid, or mixtures thereof with at least one amine of general formula III in solution at a temperature from 40 to 80° C.; and
isolating the thus prepared salts of α-lipoic acid.

26. The process of claim 24, wherein alcohols are employed as solvents.

27. The process of claim 25, wherein alcohols are employed as solvents.

28. A method of preventing skin damage, unwanted changes in the appearance of the skin, or a combination thereof comprising
applying the α-lipoic acid salt of claim 16 to the skin.

29. A method of preventing skin damage, unwanted changes in the appearance of the skin, or a combination thereof comprising applying the α-lipoic acid salt of claim 17 to the skin.

30. A composition comprising the α-lipoic acid salt of claim 16 and an excipient.

31. A composition comprising the α-lipoic acid salt of claim 17 and an excipient.

32. A composition comprising the α-lipoic acid salt of claim 16 and a formulation base.

33. A comprising the α-lipoic acid salt of claim 17 and a formulation base.

34. A composition comprising the α-lipoic acid salt of claim 16 and a carrier.

35. A composition comprising the α-lipoic acid salt of claim 17 and a carrier.

36. A composition comprising the α-lipoic acid salt of claim 16 and a diluent.

37. A composition comprising the α-lipoic acid salt of claim 17 and a diluent.
